(19) United States
(12) Patent Application Publication
(10) Pub. No.: US 2003/0165942 A1
(43) Pub. Date: Sep. 4, 2003

(54) Compound, especially marker-dye, on the basis of polymethines

(75) Inventors: Peter Czerney, Weimar (DE); Matthias Wenzel, Jena (DE); Bernd Schweder, Jena (DE); Frank Lehmann, Jena (DE)

Correspondence Address: James J. Murphy, Winstead Sechrest & Minick P.C., P.O. Box 50784, 1201 Main Street, Dallas, TX, 75250-0784, US

(73) Assignee: Dyomics GmbH, Jena (DE)

(21) Appl. No.: 10/310,206

(22) Filed: Dec. 5, 2002

(30) Foreign Application Priority Data
Dec. 5, 2001 (DE) 101 60 524.2

(51) Int. Cl. C12Q 1/68; G01N 33/53; C07D 517/02; C07D 417/02; C07D 413/02; C 07D 4 3/02

(52) U.S. Cl. 435/6; 435/7.1; 546/268.1; 548/121; 546/148; 548/159; 548/217; 548/304.4

(57) ABSTRACT

The invention relates to fluorescent dyes (fluorophores) based on polymethines for use in optical measurement and detection procedures, in particular those employing fluorescence, for example in medicine, in pharmacology and in the biological, materials and environmental sciences.

The objective was to create fluorophores based on polymethines that have a large Stokes shift, high photostability, long storage life and a high fluorescent quantum yield, and that can be excited in the simplest possible manner by white-light sources or laser radiation in the UV, visible or NIR spectral region.

According to the invention dyes on the basis of polymethines having the general formulas I, II and III

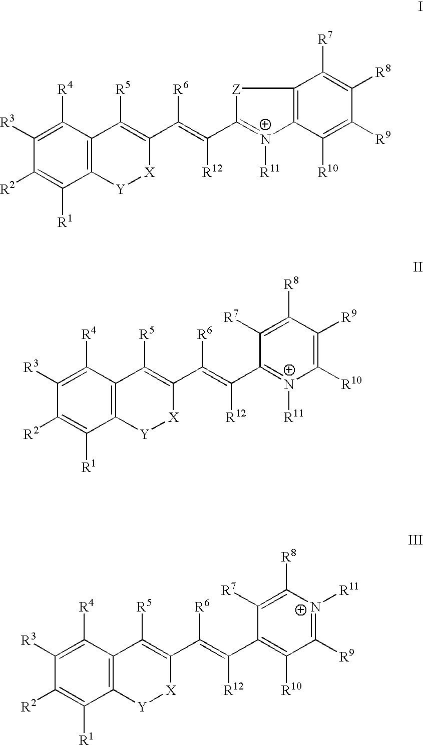

are employed.

The invention relates to fluorescent dyes (fluorophores) based on polymethines for use in optical measurement and detection procedures, in particular those employing fluorescence. Typical applications exploit the reaction of dye-labelled antigens, antibodies or DNA segments with the respective complementary species. With such methods it is possible to measure, e.g., enzyme kinetics, receptor-ligand interactions and the kinetics of nucleic-acid hybridization. Furthermore, the claimed fluorophores are of interest for the pharmacological characterization of receptors or agents.

Possible uses associated therewith exist, for example, in medicine and pharmacology, in the biological and materials sciences, in environmental monitoring and the detection of organic and inorganic microsamples present in natural surroundings and in technology.

Whereas cyanines with a Stokes shift of 20-40 nm have long been known as fluorescent markers (Cy3, Cy5, U.S. Pat. No. 5,627,027), as yet there are only a few fluorophores with a large Stokes shift. Typical examples of these are the markers derived from the laser dye DCM, with absorption maxima at 481 nm and emission maxima at 644 nm, which are claimed in the patent U.S. Pat. No. 4,886,744.

The invention is directed towards the objective of creating fluorescent markers on the basis of polymethines that have a large Stokes shift, with high photostability, long storage life and a high fluorescent quantum yield. To achieve an optimal signal-to-noise ratio, the emission bands should be in a range >520 nm, and the excitation should be achievable in the simplest possible way, by white light or laser radiation in the UV or visible spectral region. The fluorophores must have a high fluorescence output, independent of the pH value and other environmental influences. A prerequisite for covalent binding is the presence of a reactive function such that the reaction with the biomolecule to be labelled occurs under physiological conditions, or under the reaction conditions customary in the solid-phase synthesis of bio-oligomers.

The present invention describes polymethine-based marker dyes with the general formulas I or II or III:

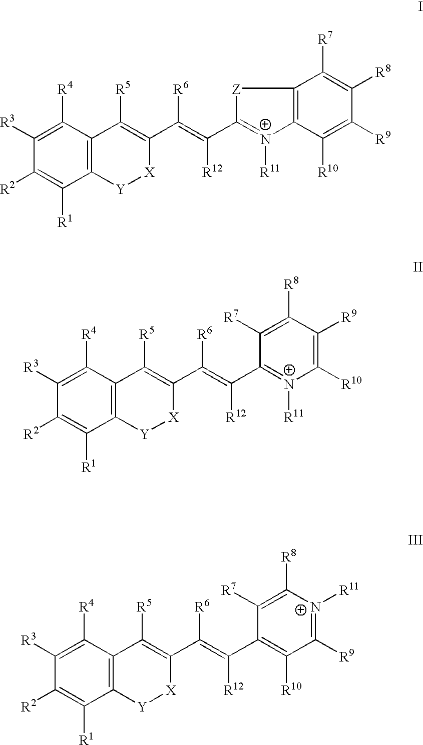

where

R¹-R¹² are the same or different and can be any of the following:

one or more chlorine and/or bromine atoms, alkyl, aryl, heteroaryl, cycloalkyl, heterocycloalkyl, alkyloxy, alkylmercapto, aryloxy, arylmercapto, heteroaryloxy, heteroarylmercapto or cyano residues,

one or more hydroxy functions,

one or more alkyl-substituted or cyclic amino functions;

and R¹ and R² and/or R³ and R⁴ and/or R⁷ and R⁸ and/or R⁹ and R¹⁰ can form additional aliphatic or aromatic rings, and/or two adjacent residues, e.g. R⁵ and R⁶, and in examples I or II R¹¹ and R¹², together can form one or more aliphatic or aromatic rings,

X—Y together can stand for an element in the group O, S, Se, Te or the structural element (CR₂),ₙ, NR or SO₂, wherein R can take on the same one or different ones of the functions of R¹ -R¹², and n can stand for 1-4,

X—Y can stand for the structural elements —CR₂—O—, —O—CR₂—, —CO—O—, —O—CO—, —CO—NR— or —NR—CO—, wherein R can take on the same one or different ones of the functions of R¹- R¹²,

one or more of the substituents R¹- R¹² can be solubilizing or ionizable or ionized substituents such as SO₃⁻, PO₃²⁻, CO₂⁻, O⁻, NR₃⁺, cyclodextrin or sugar, which determine the hydrophilic properties of the dyes, and it is also possible for these substituents to be bound to the actual chromophore by way of an aliphatic or heteroaliphatic, possibly cyclic spacer group,

at least one of the substituents R¹- R¹² stands for a reactive group of the type isocyanate, isothiocyanate, hydrazine, amine, mono- and dichloro- or mono- and dibromotriazine, aziridine, sulfonyl halogenide, N-hydroxysuccinimide ester, imido ester, glyoxal or aldehyde, or maleimide or iodacetamide and phosphoramidite, and the substituent in each case can be bound to the actual basic chromophore by way of an aliphatic or heteroaliphatic, possibly cyclic spacer group,

the aliphatic or heteroaliphatic spacer group consists of a structural element —[(CH₂)ₐ—Y—(CH₂)_{b}]_{c}—, wherein Y can be the same or different ones of the functions CR₂, O, S, SO₂, SO₂NH, NR, COO, or CONR, R can take on the functions of R¹- R¹², and a and b represent the same or different values in the range 0-18, while c represents the values from 1 to 18,

the substituted polymethine derivatives with the general formulas I-III can be used as dyes for the optical marking of proteins, nucleic acids, oligomers, DNA, RNA, biological cells, lipids, mono-, oligo- and polysaccharides, ligands, receptors, polymers, pharmaceuticals or polymer particles,

in order to serve as dyes in systems for the qualitative or quantitative determination of proteins, nucleic acids, oligomers, DNA, RNA, biological cells, lipids, polymers, pharmaceuticals or polymer particles, the substituted polymethine derivatives with the general formulas I-III are coupled by way of the functional groups to an HO—, H₂N— or HS function of the substances to be determined,

the coupling reaction of the substituted polymethine derivatives of the general formula I-III is carried out in organic or aqueous solutions,

the conjugates comprising substituted polymethine derivatives with the general formulas I-III and biomolecules exhibit fluorescent properties,

the substituted polymethine derivatives with the general formulas I-III can be employed in qualitative and quantitative optical measurement procedures, in particular those based fluorescence, including immunological tests, hybridization procedures, chromatographic or electrophoretic methods and high-throughput screening,

or the substituted polymethine derivatives with the general formulas I-III can be employed for the analysis of receptor-ligand interactions on a microarray.

The polymethines with the general formulas I-III can be used as dyes for the optical marking of organic or inorganic identification units, e.g. amino acids, peptides, proteins, antigens, haptens, enzyme substrates, enzyme cofactors, biotin, carotinoids, hormones, neurohormones, neurotransmitters, growth factors, lympholocines, lectins, toxins, carbohydrates, oligosaccharides, polysaccharides, dextrans, nucleic acids, oligonucleotides, DNA, RNA, biological cells, lipids, receptor-binding pharmaceuticals or organic or inorganic polymeric carrier materials.

This marking of the identification units can be achieved by the production of ionic interactions between the markers having the general formulas I-III and the materials to be marked.

Furthermore, it is also possible to produce a covalent bonding of the identification unit or the carrier material to the fluorophore. This coupling reaction can be carried out in aqueous or predominantly aqueous solution and preferably at room temperature. By this means a fluorescent probe (conjugate) is produced that can be used for the qualitative or quantitative determination of various biomaterials or other organic and inorganic materials.

Both the compounds with the general formulas I-III and systems derived therefrom can be employed in qualitative and quantitative optical measurement procedures, in particular those based on fluorescence, for the diagnosis of cell properties, in biosensors (point-of-care measurements), for research on the genome (DNA sequencing) and in miniaturization technologies. Typical applications can be found in cytometry and cell-sorting, fluorescence-correlation spectroscopy (FCS), in ultra-high-throughput screening (UHTS), in multicolor fluorescence in situ hybridization (FISH) and in microarrays (DNA and protein chips).

Such a microarray is a raster-like arrangement of molecules immobilized on at least one surface, which can be used to study receptor-ligand interactions. The term “raster-like arrangement” signifies more than two molecules that are different from one another, are situated within a specified area, and in that area are immobilized in different, prespecified regions with known positions.

A receptor is a molecule that has an affinity for a given ligand. Receptors can be naturally occurring or artificially produced molecules. Receptors can be used in pure form or while bound to other species. Receptors can be linked to a binding partner by covalent or non-covalent bonds, either directly or by way of particular coupling mediators.

Examples of receptors that can be detected by means of this invention include agonists and antagonists for cell-membrane receptors, toxins and other poisonous substances, viral epitopes, hormones such as opiates and steroids, hormone receptors, peptides, enzymes, enzyme substrates, agents that function as cofactors, lectins, sugars, oligonucleotides, nucleic acids, oligosaccharides, cells, cell fragments, tissue fragments, proteins and antibodies, but are not limited to the substances listed here.

A ligand is a molecule that is recognized by a particular receptor. Examples of ligands that can be detected by this invention include agonists and antagonists for cell-membrane receptors, toxins and other poisonous substances, viral epitopes, hormones such as opiates and steroids, hormone receptors, peptides, enzymes, enzyme substrates, agents that function as cofactors, lectins, sugars, oligonucleotides, nucleic acids, oligosaccharides, proteins and antibodies, but are not limited to the substances listed here.

By the preparation of asymmetrical polymethines, which comprise on one hand, as a terminal function, a readily derivatizable heterocycle of the type CH-acid compounds, and on the other hand a novel substituted 6-ring heterocycle, in particular the following advantages are achieved:

With suitable substitution, the absorption maxima are in the region of the emission wavelengths of the argon-ion laser (488 nm), and the emission maxima observed are in the range between 550 and 660 nm.

Another advantage resides in the fact that the hydrophilic character of the differently emitting fluorophores can be made nearly identical.

General directions for producing the dyes:

0.2 mmol of the substituted coumarin-3-carbaldehyde, or 0.2 mmol of the benzofuran-2-carbaldehyde, and 0.2 mmol of the corresponding CH-active compound are heated with reflux in 5 ml acetic anhydride for 10 hours. After cooling ether is added and the resulting precipitate is isolated and purified by column chromatography.

### EXEMPLARY EMBODIMENT 1

### 1-(5-carboxy-pentyl)-2-[(E)-2-(7-diethylamino-2-oxo-2H-chromen-3-yl)-vinyl]-pyridinium Bromide

0.2 mmol 7-diethylamino-2-oxo-2H-chromen-3-carbaldehyd and 0.2 mmol 1-(5-carboxy-pentyl)-2-methyl-pyridinium bromide are converted according to the general directions above. Column chromatography: SiO₂, eluent: ethanol/toluene.

35% yield, MS (ESI+): 435 (M⁺) , UV-Vis (in ethanol): λₘₐₓ 480 nm, λₑₘ 600 nm

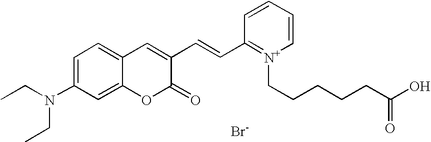

### EXEMPLARY EMBODIMENT 2

### 1-(5-carboxy-pentyl)-2-[(E)-2-(7-diethylamino-2-oxo-2H-chromen-3-yl)-vinyl]-5-sulfonato-pyridinium Betaine

0.2 mmol 7-diethylamino-2-oxo-2H-chromene-3-carbaldehyde and 0.2 mmol 1-(5-carboxy-pentyl)-2-methyl-5-sulfonato-pyridinium betaine are converted according to the general directions above. Column chromatography: SiO₂ (RP18), eluent: methanol/water.

30% yield, MS (ESI+): 537 (M+Na⁺), UV-Vis (in water): λₘₐₓ 505 nm, λₑₘ 640 nm

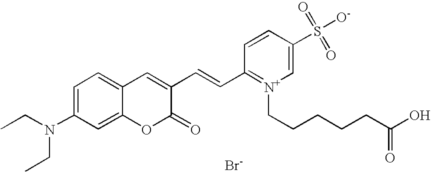

### EXEMPLARY EMBODIMENT 3

### 1-(5-carboxy-pentyl)-4-[(E)-2-(7-diethylamino-2-oxo-2H-chromen-3-yl)-vinyl]-pyridinium Bromide

0.2 mmol 7-diethylamino-2-oxo-2H-chromene-3-carbaldehyde and 0.2 mmol 1-(5-carboxy-pentyl)-4-methyl-pyridinium bromide are converted according to the general directions above. Column chromatography: SiO₂, eluent: ethanol/toluene.

42% yield, MS (ESI+): 435 (M⁺), UV-Vis (in ethanol): λₘₐₓ 500 nm, λₑₘ 630 nm

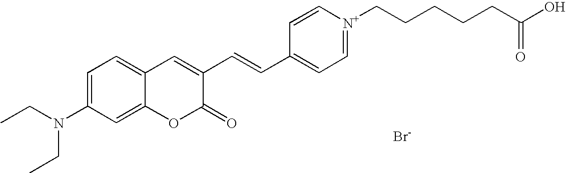

### EXEMPLARY EMBODIMENT 4

### 1-(5-carboxy-pentyl)-4-((E)-2-{7-[ethyl-(3-sulfonatopropyl)-amino]-2-oxo-2H-chromen-3-yl}-vinyl)-pyridinium Betaine

0.2 mmol 3-[ethyl-(3-formyl-2-oxo-2H-chromen-7-yl)-amino]-propane-1-sulfonic acid sodium salt and 0.2 mmol 1-(5-carboxy-pentyl)-4-methyl-pyridinium bromide are converted according to the general directions above. Column chromatography: SiO₂ (RP18), eluent: methanol/water.

28% yield, MS (ESI−): 527 (M−H⁺), UV-Vis (in water): λₘₐₓ 480 nm, λₑₘ 624 nm

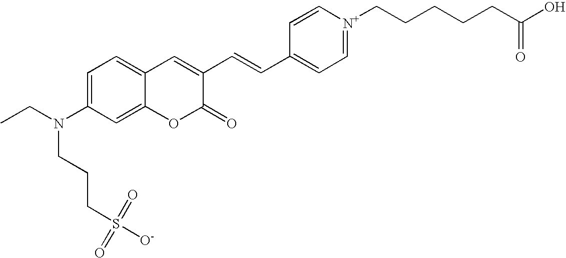

### EXEMPLARY EMBODIMENT 5

### 1-(5-carboxy-pentyl)-4-[(E)-2-(7-diethylamino-2-oxo-2H-chromen-3-yl)-vinyl]-3-sulfonato-pyridinium Betaine

0.2 mmol 7-diethylamino-2-oxo-2H-chromene-3-carbaldehyde and 0.2 mmol 1-(5-carboxy-pentyl)-3-sulfonato-4-methyl-pyridinium betaine are converted according to the general directions above. Column chromatography: SiO₂ (RP18), eluent: methanol/water.

30% yield, MS (ESI+): 537 (M−Na⁺), UV-Vis (in water): λₘₐₓ 524 nm, λₑₘ 660 nm

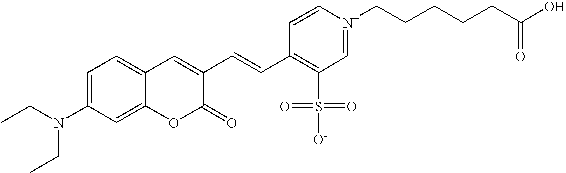

### EXEMPLARY EMBODIMENT 6

### 1-(5-carboxy-pentyl)-4-[(E)-2-(7-diethylamino-4-methoxy-2-oxo-2H-chromen-3-yl)-vinyl]-3-sulfonato-pyridinium Betaine

0.2 mmol 7-diethylamino-4-(1-morpholino)-2-oxo-2H-chromene-3-carbaldehyde and 0.2 mmol 1-(5-carboxy-pentyl)-3-sulfonato-4-methyl-pyridinium betaine are converted in methanol in the presence of triethylamine. Column chromatography: SiO₂ (RP18), eluent: methanol/water.

10% yield, MS (ESI+): 567 (M−Na⁺), UV-Vis (in water): λₘₐₓ 505 nm, λₑₘ 645 nm

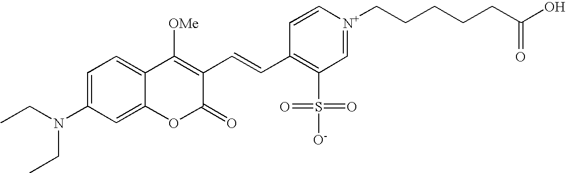

### EXEMPLARY EMBODIMENT 7

### 1-(5-carboxy-pentyl)-4-[(E)-2-(7-diethylamino-4-hydroxy-2-oxo-2H-chromen-3-yl)-vinyl]-3-sulfonato-pyridinium Betaine

0.2 mmol 7-diethylamino-4-(1-morpholino)-2-oxo-2H-chromene-3-carbaldehyde and 0.2 mmol 1-(5-carboxy-pentyl)-3-sulfonato-4-methyl-pyridinium betaine are converted in a methanol/water mixture in the presence of triethylamine. Column chromatography: SiO₂ (RP18), eluent: methanol/water.

15% yield, MS (ESI−): 529 (M−H⁺), UV-Vis (in water): λₘₐₓ 500 nm, λₑₘ 590 nm

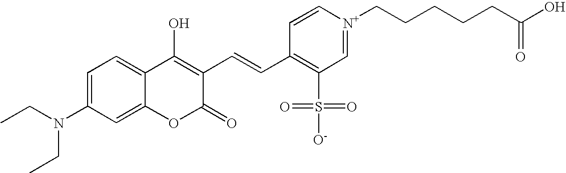

### EXEMPLARY EMBODIMENT 8

### 1-(5-carboxy-pentyl)-2-[(E)-2-(7-diethylamino-4-hydroxy-2-oxo-2H-chromen-3-yl)-vinyl]-3,3-dimethyl-5-sulfonato-3H-indolium Betaine

0.2 mmol 7-diethylamino-4-hydroxy-2-oxo-2H-chromene and 0.2 mmol 1-(5-carboxy-pentyl)-3,3-dimethyl-2-((E)-2-phenylamino-vinyl)-5-sulfonato-3H-indolium betaine are converted according to the general directions given above. Column chromatography: SiO₂ (RP18), eluent: methanol/water.

35% yield, MS (ESI−): 595 (M−H⁺), UV-Vis (in water): λₘₐₓ 505 nm, λₑₘ 600 nm

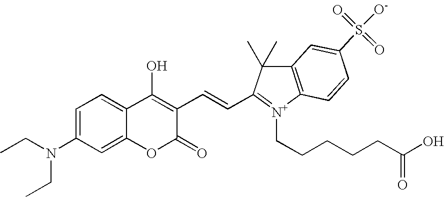

### EXEMPLARY EMBODIMENT 9

### 1-(5-carboxy-pentyl)-4-[(E)-2-(7-diethylamino-2-oxo-2H-chromen-3-yl)-vinyl]-chinolinium Bromide

0.2 mmol 7-diethylamino-2-oxo-2H-chromene-3-carbaldehyde and 0.2 mmol 1-(5-carboxy-pentyl)-4-methyl-chinolinium bromide are converted according to the general directions given above. Column chromatography: SiO₂, eluent: ethanol/toluene.

30% yield, MS (ESI+): 485 (M⁺) , UV-Vis (in methanol): λₘₐₓ 540 nm, λₑₘ 695 nm

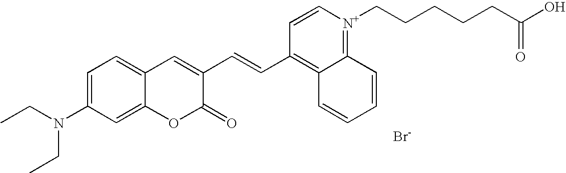

### EXEMPLARY EMBODIMENT 10

### 1-(5-carboxy-pentyl)-4-[(E)-2-(7-diethylamino-2-oxo-2H-chromen-3-yl)-vinyl]-6-sulfo-chinolinium Betaine

0.2 mmol 7-diethylamino-2-oxo-2H-chromene-3-carbaldehyde and 0.2 mmol 1-(5-carboxy-pentyl)-4-methyl-6-sulfonato-chinolinium betaine are converted according to the general directions given above. Column chromatography: SiO₂ (RP18), eluent: methanol/water.

20% yield, MS (ESI+): 465 (M+H⁺) and 587 (M+Na⁺), UV-Vis (in methanol): λₘₐₓ 555 nm, λₑₘ 715 nm

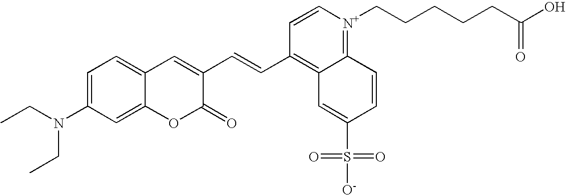

### EXEMPLARY EMBODIMENT 11

### 1-(5-carboxy-pentyl)-2-[(E)-2-(7-diethylamino-2-oxo-2H-chromen- 3-yl)-vinyl]-6-methyl-chinolinium Bromide

0.2 mmol 7-diethylamino-2-oxo-2H-chromene-3-carbaldehyde and 0.2 mmol 1-(5-carboxy-pentyl)-4,6-dimethyl-chinolinium bromide are converted according to the general directions given above. Column chromatography: SiO₂ (RP18), eluent: methanol/water.

10% yield, MS (ESI+): 499 (M⁺), UV-Vis (in methanol): λₘₐₓ 520 nm, λₑₘ 655 nm

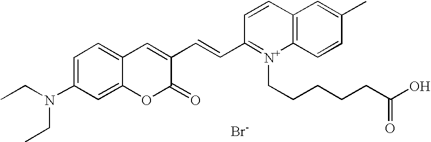

### EXEMPLARY EMBODIMENT 12

### 1-(5-carboxy-pentyl)-2-[(E)-2-(6-diethylamino-benzofuran-2-yl)-vinyl]-5-sulfonato-pyridinium Betaine

0.2 mmol 6-diethylamino-benzofuran-2-carbaldehyde and 0.2 mmol 1-(5-carboxy-pentyl)-2-methyl-5-sulfonato-pyridinium betaine are converted according to the general directions given above. Column chromatography: SiO₂ (RP18), eluent: methanol/water.

10% yield, MS (ESI+): 487 (M+H⁺) and 509 (M+Na⁺), UV-Vis (in water): λₘₐₓ 560 nm, λₑₘ 690 nm

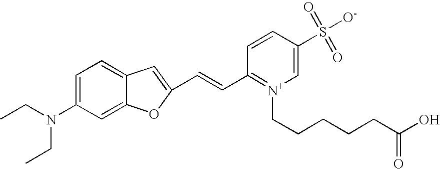

### EXEMPLARY EMBODIMENT 13

### 1-(5-carboxy-pentyl)-4-[(E)-2-(6-diethylamino-benzofuran-2-yl)-vinyl]-3-sulfonato-pyridinium Betaine

0.2 mmol 6-diethylamino-benzofuran-2-carbaldehyde and 0.2 mmol 1-(5-carboxy-pentyl)-4-methyl-3-sulfonato-pyridinium betaine are converted according to the general directions given above. Column chromatography: SiO₂ (RP18), eluent: methanol/water.

15% yield, MS (ESI+): 487 (M+H⁺) and 509 (M+Na⁺), UV-Vis (in water): λₘₐₓ 530 nm, λₑₘ 730 nm

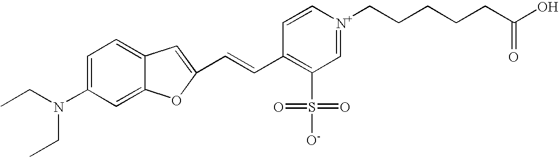

### EXEMPLARY EMBODIMENT 14

### 1-(5-carboxy-pentyl)-4-[(E)-2-(6-diethylamino-benzofuran-2-yl)-vinyl]-6-sulfonato-chinolinium Betaine

0.2 mmol 6-diethylamino-benzofuran-2-carbaldehyde and 0.2 mmol 1-(5-carboxy-pentyl)-4-methyl-6-sulfonato-chinolinium betaine are converted according to the general directions given above. Column chromatography: SiO₂ (RP18), eluent: methanol/water.

18% yield, MS (ESI+): 537 (M+H⁺) and 559 (M+Na⁺), UV-Vis (in methanol): λₘₐₓ 630 nm, λₑₘ 800 nm

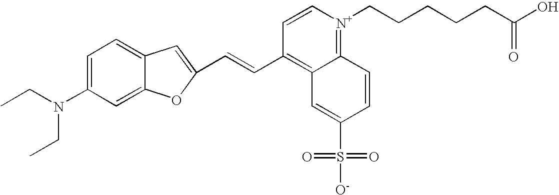

### EXEMPLARY EMBODIMENT 15

### 1- (5-carboxy-pentyl) -2-[(E)-2- (6-diethylamino-benzofuran-2-yl)-vinyl]-3,3-dimethyl-5-sulfonato-3H-indolium Betaine

0.2 mmol 6-diethylamino-benzofuran-2-carbaldehyde and 0.2 mmol 1-(5-carboxy-pentyl)-2,3,3-trimethyl-5-sulfonato-3H-indolium betaine are converted according to the general directions given above. Column chromatography: SiO₂ (RP18), eluent: methanol/water.

12% yield, MS (ESI+): 553 (M+H⁺) and 575 (M+Na⁺), UV-Vis (in water): λₘₐₓ 650 nm, λₑₘ 715 nm

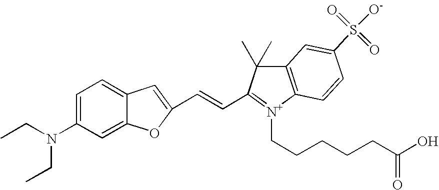

1. Compound, in particular marker-dye, on the basis of polymethines having the general formulas I-III 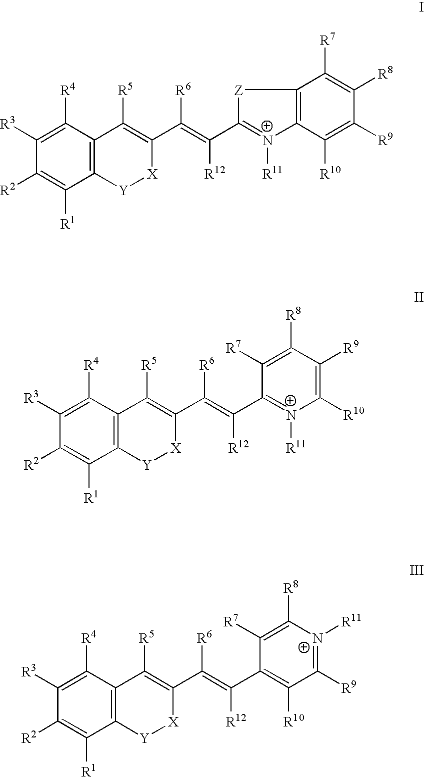
where
R¹-R¹² are the same or different and can be any of the following:
one or more chlorine and/or bromine atoms, alkyl, aryl, heteroaryl, cycloalkyl, heterocycloalkyl, alkyloxy, alkylmercapto, aryloxy, arylmercapto, heteroaryloxy, heteroarylmercapto or cyano residues,
one or more hydroxy functions,
one or more alkyl-substituted or cyclic amino functions;
and R¹ and R² and/or R³ and R⁴ and/or R⁷ and R⁸ and/or R⁹ and R¹⁰ can form additional aliphatic or aromatic rings, and/or two adjacent residues, e.g. R⁵ and R⁶, and in examples I or II R¹¹ and R¹², together can form one or more aliphatic or aromatic rings,
X—Y together can stand for an element in the group O, S, Se, Te or the structural element (CR₂)ₙ, NR or SO₂, wherein R can take on the same one or different ones of the functions of R¹- R¹², and n can stand for 1-4,
X—Y can stand for the structural elements —CR₂—O—, —O—CR₂—, —CO—O, —O—CO—, —CO—NR— or —NR—CO—, wherein R can take on the same one or different ones of the functions of R¹- R¹².

2. Compound according to claim 1, characterized in that one or more of the substituents R¹-R¹² can be solubilizing or ionizable or ionized substituents such as SO₃⁻, PO₃²⁻, CO₂⁻, O⁻, NR₃⁺, cyclodextrin or sugar, which determine the hydrophilic properties of the dyes, and it is also possible for these substituents to be bound to the actual chromophore by way of an aliphatic or heteroaliphatic or cyclic spacer group.

3. Compound according to claims 1 and 2, characterized in that at least one of the substituents R¹-R¹² stands for a reactive group of the type isocyanate, isothiocyanate, hydrazine, amine, mono- and dichloro- or mono- and dibromotriazine, aziridine, sulfonyl halogenide, N-hydroxysuccinimide ester, imido ester, glyoxal or aldehyde or maleimide or iodoacetamide and phosphoramidite, and the substituent in each case can be bound to the actual basic chromophore by way of an aliphatic, heteroaliphatic, or cyclic spacer group.

4. Compound according to claims 1 to 3, characterized in that the aliphatic or heteroaliphatic spacer group consists of a structural element —[(CH₂)ₐ—Y—(CH₂)_{b}]_{c}—, wherein Y can be the same or different ones of the functions CR₂, O, S, SO₂, SO₂NH, NR, CO₂, or CONR, R can take on the functions of R¹- R¹², and a and b represent the same or different values in the range 0-18, while c represents the values from 1 to 18.

5. Application of the substituted polymethine derivatives having the general formulas I-III as dyes for the optical marking of proteins, nucleic acids, oligomers, DNA, RNA, biological cells, lipids, mono-, oligo- and polysaccharides, ligands, receptors, polymers, pharmaceuticals or polymer particles.

6. System for the qualitative or quantitative determination of proteins, nucleic acids, oligomers, DNA, RNA, biological cells, lipids, polymers, pharmaceuticals or polymer particles, characterized in that the functional groups of the compounds according to claims 1 to 4 can be coupled by covalent bonds to a HO, H₂N or HS function of the substances to be determined.

7. System according to claim 6, characterized in that the coupling reaction is carried out in organic or aqueous solutions.

8. System according to claims 6 and 7, characterized in that the covalently coupled compound exhibits fluorescent properties.

9. Application of the compounds and systems according to claims 1 to 8 in qualitative and quantitative optical, in particular fluorescence-optical measurement procedures including immunological tests, hybridization methods, DNA sequencing, chromatographic or electrophoretic methods and in high-throughput screening.

10. Application of the compounds and systems mentioned in claims 1 to 8 for the analysis of receptor-ligand interactions on a microarray.
